(19) United States
(12) Patent Application Publication
(10) Pub. No.: US 2024/0287563 A1
(43) Pub. Date: Aug. 29, 2024

(54) METHOD FOR PRODUCING BETA-1,3-1,6-GLUCAN

(71) Applicants: Sophy Inc., Kochi (JP); NATIONAL UNIVERSITY CORPORATION KOCHI UNIVERSITY, Kochi (JP)

(72) Inventors: Yasunori IKEUE, Kochi (JP); Mitsuru NAGATAKI, Kochi (JP); Shinji NAGATA, Kochi (JP)

(21) Appl. No.: 18/561,083

(22) PCT Filed: May 23, 2022
(86) PCT No.: PCT/JP2022/021101

§ 371 Date: Nov. 15, 2023

(30) Foreign Application Priority Data
May 24, 2021 (JP) 2021-087255

(51) Int. Cl. C12P 19/04; C08B 37/00; C12R 1/01

(52) U.S. Cl. CPC C12P 19/04; C08B 37/0024; C12R 2001/01

(57) ABSTRACT

Methods for producing a novel β-1,3-1,6-glucan exhibiting an excellent anticancer action are provided. The methods can include using a novel black yeast-like bacteria is used in producing the β-1,3-1,6-glucan and culturing an Aureobasidium pullulans APNN-M 163 strain (accession number: NITE BP-03377) in a culture medium.

### CROSS-REFERENCE TO RELATED APPLICATIONS

This application claims priority to International Application No. PCT/JP2022/021101, filed May 23, 2022, and claims the benefit of Japanese Application No. 2021-087255, filed May 24, 2021, each application of which is hereby incorporated herein by reference in its entirety.

### BACKGROUND

### Field of Invention

The present invention relates to: a method for producing novel β-1,3-1,6-glucan exhibiting an excellent anticancer action; novel black yeast-like bacteria used in said production method; and novel β-1,3-1,6-glucan exhibiting an excellent anticancer action.

### Background Art

β-glucan is a general term for a polymer of glucose bound with β-glucoside bond, generally D-glucose bound with β-1,3-bond, while β-glucans produced by yeast and fungi have β-1,6-bond branches and cereal β-glucans have β-1,3-bonds and β-1,4-bond skeletons. β-glucan is a kind of insoluble dietary fiber which, like other dietary fibers, is known that helps preventing constipation and lifestyle-related disease, but its preventive and ameliorative effect against cancer and immunostimulatory action are attracting attention. In particular, β-glucan derived from basidiomycetes is well known for its anticancer and immunostimulatory actions, but β-glucan is used as a constituent of cell walls and is bound to protein, it is considered to be difficult to purify β-glucan from basidiomycetes. On the other hand, black yeast-like bacteria (Aureobasidium pullulans) can release produced β-glucan outside the bacterial cell, so they are used as a producer being able to efficiently produce high-purity β-glucan.

For example, in Patent Document 1, production of β-1,3-1,6-glucan using genus Aureobasidium strain, and FO-68 strain (AFO-202 strain) are described. In addition, in Patent Document 2, Aureobasidium pullulans M-3 strain as a microorganism for efficient production of β-glucan is disclosed.

### PRIOR ART DOCUMENTS

### Patent Documents

[Patent Document 1] Japanese Unexamined Patent Application Publication No. 2004-329077.
[Patent Document 2] Japanese Unexamined Patent Application Publication No. 2020-31580.

### SUMMARY OF THE INVENTION

### Problems to be Solved by Invention

As described above, especially β-glucans derived from fungi are known to have anticancer and immunostimulatory actions, but β-glucans have different types in chemical structure such as differences in branching structure as well as in molecular weight and water solubility, and they vary in each action and its strength.

Then, the purpose of the present invention is to provide: a method for producing novel 3-1,3-1,6-glucan exhibiting an excellent anticancer action; novel black yeast-like bacteria used in said production method; and novel β-1,3-1,6-glucan exhibiting an excellent anticancer action.

### Means of Solving Problems

The inventors conducted intensive research to solve the above problems. As a result, a black yeast-like bacteria producing a novel β-1,3-1,6-glucan by mutation treatment was created, and it was found that such β-1,3-1,6-glucan exhibits an excellent anticancer action to complete the invention.

The present invention is described below:
[1] A method for producing β-1,3-1,6-glucan, characterized by comprising a step for culturing an Aureobasidium pullulans APNN-M163 strain (accession number: NITE BP-03377) in a culture medium.
[2] The method according to said [1], wherein the culture medium contains one or more carbon sources selected from glucose, polysaccharides containing glucose unit, and fructose.
[3] The method according to said [1] or [2], wherein the culture medium contains nitrate ion salt as a nitrogen source.
[4] Aureobasidium pullulans APNN-M163 strain (accession number: NITE BP-03377).
[5] β-1,3-1,6-glucan characterized by inclusion of β-1,4 and β-1,2 bonds in addition to β-1,3 and β-1,6 bonds.
[6] The β-1,3-1,6-glucan according to said [5], wherein the ratio of glucose units bound to adjacent glucose units at only 1st and 4th positions to total glucose units configuring the β-1,3-1,6-glucan is 0.04 or more and 0.08 or less.
[7] The β-1,3-1,6-glucan according to said [5] or [6], wherein the ratio of glucose units bound to adjacent glucose units at 1st, 2nd, 4th and 6th positions to total glucose units configuring the β-1,3-1,6-glucan is 0.04 or more and 0.08 or less.

### Advantageous Effects of Invention

The present invention can provide a novel β-1,3-1,6-glucan that exhibits an excellent anticancer action. In addition, the present invention also provides a novel black yeast-like bacteria that produces such novel β-1,3-1,6-glucan and releases it outside the bacterial cell. Therefore, the present invention is extremely excellent industrially as related to novel β-1,3-1,6-glucan itself, which is useful as an active ingredient in health foods, etc., and its production technology.

### BRIEF DESCRIPTION OF THE DRAWING

FIG. 1 shows adhesive properties of β-glucan produced by M163 strain according to the present invention and the one produced by AFO-202 strain.

### DETAILED DESCRIPTION

### Embodiments for Carrying Out Invention

A method for producing β-1,3-1,6-glucan according to the present invention comprises a step for culturing Aureobasidium pullulans APNN-M163 strain (accession number: NITE BP-03377) in a liquid culture medium.

The Aureobasidium pullulans APNN-M163 strain (which hereinafter may be abbreviated as “M163 strain”), a black yeast-like bacteria according to the present invention has been internationally deposited with a depository institution as follows: (i) Name and address of the depository institution
Name: National Institute of Technology and Evaluation, Patent Microorganisms Depositary (NPMD)
Address: No. 122, 2-5-8, Kazusakamatari, Kisarazu-shi, Chiba, Japan
(ii) Date of original deposit: Feb. 9, 2021
(iii) Transfer Date: Aug. 16, 2021
(iv) Accession number: NITE BP-03377

M163 strain is a dimorphic fungus that grows in sole yeast form as well as in mycelial form. When it is cultured at 25° C. on solid culture medium, it forms white to yellowish-white colored opaque circular colony, and the colony grows to about 2 cm in diameter in a week and 3 cm or larger in diameter in two weeks. The periphery of the colony is wavy, flatly raised, and mucoid is visible on the surface. The initial pH of the culture medium is preferably 7.0+/−0.2, but as the culture progresses, the pH of the culture medium decreases, and at pH 3.5 or lower, the bacteria aggregate with each other, making it difficult to produce β-glucan. Therefore, it is preferable to adjust the pH of the culture medium in a timely manner if the culturing time gets longer.

Based on the base sequence information of 18S rDNA gene of M163 strain, etc., it has been concluded that M163 strain is a black yeast-like bacteria classified as Aureobasidium pullulans.

M163 strain produces novel β-1,3-1,6-glucan according to the present invention by culturing and releases it outside the bacterial cell. Purification of said β-1,3-1,6-glucan is easier if liquid culture medium is used as the culture medium for culturing. In addition, said β-1,3-1,6-glucan is obviously different from conventional β-1,3-1,6-glucan, and is useful as an active ingredient in health foods, etc., because it exhibits more excellent immunostimulatory action.

When producing β-1,3-1,6-glucan according to the present invention using M163 strain, it is preferable to add one or more carbon sources selected from glucose, polysaccharides containing glucose unit and fructose to the culture medium. The polysaccharides containing glucose unit include disaccharide as long as it is a polysaccharide containing glucose as a constitutional unit, and also include, for example, disaccharides such as sucrose, lactose, maltose, trehalose, cellobiose; trisaccharides such as nigerotriose, maltotriose, melezitose, maltotriulose, raffinose, kestose; tetrasaccharides such as nystose, nigerotetraose, stachyose; starch and glycogen, etc. In addition, fructose can also be used, because it may be converted to glucose by isomerase. D-glucose is preferable as a carbon source. The ratio of the carbon source in the culture medium may be adjusted appropriately within the range where the M163 strain grows well and β-1,3-1,6-glucan is produced well, and it can be, for example, 0.5% or more by mass and 5% or less by mass, it is preferably 1% or more by mass and 2% or less by mass.

Nitrogen sources for the culture medium include, for example, inorganic nitrogen sources such as nitrate ion salt and ammonium salt; and organic nitrogen sources such as amino acid, peptone, triptone, and casamino acid. In the present invention, from the viewpoint of production efficiency of β-1,3-1,6-glucan by M163 strain, it is preferable that at least nitrate ion salt to be blended into the culture medium. The nitrate ion salts added to the culture medium as the nitrogen source include, for example, thiamine nitrate, potassium nitrate, calcium nitrate, and ammonium nitrate, etc., and in the present invention, using thiamine nitrate is preferable because it is safe to be used for foods. The ratio of the nitrogen source in the culture medium may be adjusted appropriately within the range where the M163 strain grows well and β-1,3-1,6-glucan is produced well, and it can be, for example, 0.005% or more by mass and 1% or less by mass, it is preferably 0.01% or more by mass and 0.12% or less by mass.

Further, general culture medium ingredients are blended in the culture medium. Other culture medium ingredients include, for example, inorganic salts such as sodium chloride, potassium chloride, and calcium chloride; metallic components such as magnesium, zinc, copper, and selenium; and vitamins such as vitamin B, vitamin A, vitamin E, riboflavin, thiamine, and biotin. Extracts such as yeast extract, meat extract, and plant extracts such as potato extract, and peptone such as proteose peptone, myocardium peptone, gelatin peptone, soy peptone may be blended as a culture medium ingredient that contains above ingredients compositely. In addition, antifoaming agent may be added.

Culturing conditions may be adjusted appropriately within the range where the M163 strain grows well and β-1,3-1,6-glucan is produced well. For example, the pH of the culture medium is preferably 6.5 or higher and 7.5 or lower, and more preferably 6.8 or higher and 7.2 or lower, but as mentioned above, the pH of the culture medium decreases as the culture of the M163 strain progresses, so it is preferable that the pH of the culture medium is adjusted appropriately. In addition, culture temperature is preferably 20° C. or higher and 30° C. or lower, and more preferably 25° C.+/−2° C. The M163 strain may be cultured statically, but shaking, stirred, or rotation culture is preferred. As for culturing time, culture may be carried out until the M163 strain grows sufficiently and sufficient amount of β-1,3-1,6-glucan is produced, it can be, for example, 10 hours or longer and 100 hours or shorter.

After culturing the M163 strain and producing β-1,3-1,6-glucan, β-1,3-1,6-glucan may be purified by conventional method. For example, after the culture, solid component in the culture solution may be removed with centrifugation or filtration, ethanol may be added to obtained supernatant to precipitate β-1,3-1,6-glucan, and liquid content may be removed with centrifugation or filtration, subsequently it may be dried.

β-1,3-1,6-glucan according to the present invention includes β-1,4 and β-1,2 bonds in addition to β-1,3 and β-1,6 bonds. General β-glucan is configured with polymer in which glucose is bound by 1,3-glycosidic bond as basic skeleton, in addition, β-1,4 bond can be found in plant-derived β-glucan, and side chain with β-1,6 bonds can be found in fungi-derived β-glucan. The β-1,3-1,6-glucan according to the present invention, although it is yeast-derived β-glucan, has a unique chemical structure that, in addition to main chain with β-1,3 bond and side chain with β-1,6 bond, contains glucose units bound to adjacent glucose unit via β-1,4 bond and also contains glucose units bound to adjacent glucose units via β-1,3, β-1,6 and β-1,2 bonds.

The ratio of glucose units bound to adjacent glucose units at only 1st and 4th positions, which is characteristic in the β-1,3-1,6-glucan according to the present invention, to total glucose units configuring β-1,3-1,6-glucan is preferably 0.04 or more and 0.08 or less. In addition, the ratio of glucose units bound to adjacent glucose units at 1st, 2nd, 4th and 6th positions, which is characteristic in the β-1,3-1,6-glucan according to the present invention, to total glucose units configuring β-1,3-1,6-glucan is preferably 0.04 or more and 0.08 or less.

Chemical structural formulas of glucose unit bound to adjacent glucose unit at only 1st and 4th positions, and glucose unit bound to adjacent glucose unit at 1st, 2nd, 4th and 6th positions are shown as per below. In the formula, end of the bound unit shall be bound to adjacent glucose unit.

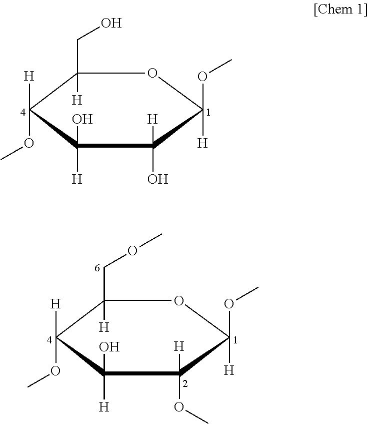

Further, composition of the glucose that constitutes the β-1,3-1,6-glucan according to the present invention can be determined by conventional method. That is, the hydroxyl groups present in the β-1,3-1,6-glucan are completely methylated, subsequently resulting composition is hydrolyzed to methylated glucose. Then, partially methylated acetyl derivative of sorbitol is obtained by reducingly acetylating hydroxyl group generated by the hydrolysis. Binding mode of each glucose can be estimated by analyzing obtained acetyl derivative with gas chromatography and GC-MS.

The β-1,3-1,6-glucan according to the present invention exhibits significantly higher adhesive property than conventional β-1,3-1,6-glucan and is obviously different from conventional β-1,3-1,6-glucan. In addition, the β-1,3-1,6-glucan according to the present invention exhibits an excellent anticancer action. Therefore, the β-1,3-1,6-glucan according to the present invention is extremely useful as an active ingredient for health foods.

The present application claims the benefit of priority based on Japanese Patent Application No. 2021-87255 filed on May 24, 2021. Entire contents of specification of Japanese Patent Application No. 2021-87255 filed May 24, 2021, are hereby incorporated by reference in the present application.

### EXAMPLES

The present invention is described more specifically below with examples, however the present invention is never limited by the following examples, and, of course, can be carried out with appropriate alterations within the range that it conforms to the purposes described above and below, all of which are included in the technical scope of the present invention.

### Example 1: APNN-M163 Strain

### (1) Obtaining APNN-M163 Strain

Plant and starch-rich soil sample was collected, suspended in sterile water, applied to PDA culture medium (2.4 w/v % of potato dextrose (made by BD Difco), 1.5 w/v % of agar, pH 5.2) or 1/6 PDA culture medium (0.4 w/v % of potato dextrose (made by BD Difco), 2.0 w/v % of glucose, 1.5 w/v % of agar, pH 5.2), and cultured at 30° C. Bacteria in which viscous substance is confirmed on surface of colony was isolated and cultured statically for one week at 20° C. with a small amount of synthetic culture medium (0.06 w/v % of (NH₄)₂SO₄, 0.2 w/v % of KH₂PO₄, 0.02 w/v % of MgSO₄-7H₂O, 0.01 w/v % of NaCl, 0.04 w/v % of yeast extract (made by Oriental Yeast Co., ltd.), pH 5.2) or rice bran culture medium (0.2 w/v % of rice bran, 0.2 w/v % of ascorbic acid, 1.0 w/v % of sucrose, pH 5.2). The culture solution was centrifuged at about 10,000 g for 10 minutes, and strains were sorted by measuring amount of polysaccharide contained in obtained supernatant. For measuring the amount of polysaccharide, ethanol was added so the final concentration to be 70%, and phenol sulfuric acid method was used to measure total sugar amount of the precipitate.

The selected strains were mutation treated. General techniques such as ethyl methanesulfonate (EMS), UV, and nitrous acid were used for mutation treatment. Mutation-treated strains were also sorted by measuring the amount of polysaccharide in the supernatant, as were the parent strains.

The sorted strains were cultured, and useful bacteria were sorted according to type and amount of polysaccharides produced by the bacteria. Conditions are as follows. One inoculating loop of bacteria cultured for 4 days at 25° C. using PDA culture medium were inoculated to 100 mL flask containing 30 mL of rice bran culture medium or synthetic culture medium, and cultured at 25° C. for 48 hours with shaking at 125 rpm. 1 v/v % of obtained pre-cultured solution was inoculated into a 1 L flask containing 350 mL of rice bran culture medium or synthetic culture medium, and cultured at 25° C., for 72 hours with rotation at 180 rpm to obtain main culture solution.

The main culture solution was centrifuged at about 10,000 g for 10 minutes, and ethanol was added to obtained supernatant so the final concentration to be 70%, and amount of polysaccharide was measured by measuring total sugar amount of the precipitate with phenol sulfuric acid method. Type of polysaccharide was estimated by analyzing degradation products of acid hydrolysis of polysaccharides using sulfuric acid and degradation products by enzymes such as β-1,3-glucanase, β-1,6-glucanase, cellulase which decompose β-glucoside bonds, and pullulanase and amyloglucosidase which decompose α-glucoside bonds. APNN-M163 strain was isolated as a strain producing more β-glucan.

### (2) Determination of β-Glucan Structure

### (2-1) Sugar Composition Analysis

A β-glucan sample produced by APNN-M163 strain was hydrolyzed with TFA (trifluoroacetic acid) to prepare a sample solution for measurement. However, when it was re-dissolved after hydrolysis, insoluble substance remained. Since β-1,4 bond glucose is not hydrolyzed by TFA, sulfuric acid that can hydrolyze β-1,4 bond glucose was used for hydrolysis. TFA-hydrolyzed sample and sulfuric acid-hydrolyzed sample were analyzed by HPLC to determine concentration of neutral sugar in the sample solutions, and neutral sugar content in the sample solutions was calculated.

As a result, the main sugar contained in the samples was glucose. Other minor components included rhamnose, mannose, and also galactose. Glucose content contained in the TFA-hydrolyzed sample was 355 μg/mg, while the one in the sulfuric acid-hydrolyzed sample was 816 μg/mg, and neutral sugar content in the samples including components that could not be hydrolyzed by TFA was obtained by sulfuric acid hydrolysis.

### (2-2) Methylation Analysis

3-glucan sample was completely methylated, and decomposed into methylated monosaccharides by hydrolysis, which were subsequently reducingly acetylated to acetyl derivatives of partially methylated sugar alcohols (partially methylated alditol acetate). Obtained partially methylated alditol acetate was analyzed by GC and GC-MS. Obtained mass spectrum was compared with standard mass spectrum of partially methylated alditol acetate (source: Biochemical Data Book) to estimate binding mode.

Binding modes of three major mass spectrum peaks were estimated to be Glc1->, ->3Glc1->, and ->3,6Glc1->, respectively. Other than above, it was estimated that binding modes of ->4Glc1->, ->6Glc1->, and ->2,3,6Glc1-> were also included. Here, “Glc1->” represents a terminal glucose group bound to other glucose via only 1st position hydroxyl group, and, for example, “->3Glc1->” represents a glucose group bound to other two glucose groups via 3rd position and 1st position hydroxyl groups.

The most abundant binding mode included are Glc1-> and ->3,6Glc1->, and since composition ratio of them is almost 1:1 and other binding modes are also included, the β-glucan produced by APNN-M163 strain was estimated to have following chemical structure.

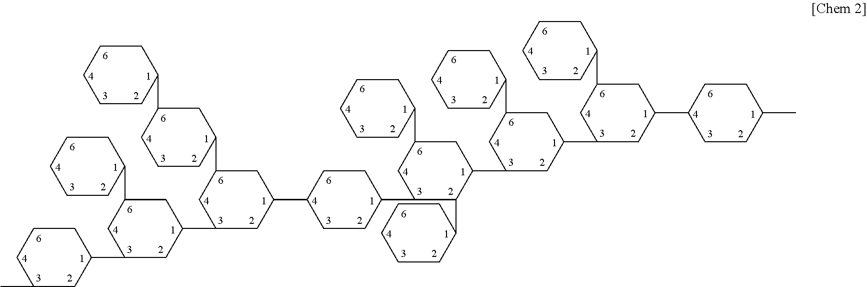

TABLE 1

| Biding position(s) | Ratio |
|---|---|
| Glc 1−> | 6 |
| −>3 Glc 1−> | 2 |
| −>4 Glc 1−> | 1 |
| −>6 Glc 1−> | 1 |
| −>3, 6 Glc 1−> | 4 |
| −>2, 3, 6 Glc 1−> | 1 |

### (3) Identification of Bacteria

Based on base sequence of 18S rDNA of APNN-M163 strain, it has been identified that APNN-M163 strain belonging to Aureobasidium pullulans.

### (4) Production of β-Glucan

APNN-M163 strain was inoculated on PDA plating medium and cultured at 25° C. for 5 to 7 days. Then, it was inoculated in a culture medium containing 60 ml of manufacturing water, 1.2 g of hydrous D-glucose, 60 mg of sodium chloride, 36 mg of sodium nitrate, 24 mg of yeast extract, 12 mg of crystalline magnesium sulfate, and 30 mg of phosphate, adjusted pH 6.8 to 7.2 using 5% sodium hydroxide aqueous solution and 25% hydrochloric acid, which was cultured at 25° C. for 48 hours with shaking at 125 rpm as a pre-culture. A culture medium with the same composition was prepared, in which 3 mL of pre-cultured solution was added, and cultured at 25° C. for 96 hours with shaking at 125 rpm.

The culture solution was centrifuged, and ethanol was added to obtained supernatant so the final concentration to be 70% or more to precipitate polysaccharides, subsequently, which was centrifuged again, and lyophilized to obtain β-glucan.

### Example 2

### (1) Production of Inoculum

APNN-M163 strain was inoculated on PDA plating medium and cultured at 25° C. for 5 to 7 days. Then, it was inoculated in a liquid culture medium and cultured at 25° C. for 48 hours with shaking at 125 rpm. Subsequently, a flask in which bacteria grew well was selected, and bacteria from well grown part was further inoculated into a liquid culture medium, and was cultured at 25° C. for 72 hours with shaking at 125 rpm.

### (2) Primary Culture

400 mL of the inoculum liquid was added into a culture medium containing 300 L of manufacturing water, 6.0 kg of hydrous D-glucose, 0.15 kg of sodium chloride, 0.18 kg of thiamine mononitrate, 0.12 kg of yeast extract, 0.03 kg of crystalline magnesium sulfate, and 0.03 L of food-grade defoaming agent, adjusted pH 6.8 to 7.2 using 5% sodium hydroxide aqueous solution and 25% hydrochloric acid, which was cultured aerated and agitated for 48 hours.

### (3) Secondary Culture

100 L of the primary culture liquid was added into a culture medium containing 5200 L of manufacturing water, 104 kg of hydrous D-glucose, 2.6 kg of sodium chloride, 3.12 kg of thiamine mononitrate, 2.08 kg of yeast extract, 0.52 kg of crystalline magnesium sulfate, and 0.52 L of food-grade defoaming agent, adjusted pH 6.8 to 7.2 using 5% sodium hydroxide aqueous solution and 25% hydrochloric acid, which was cultured aerated and agitated for 72 hours.

### (4) Purification of β-Glucan

The secondary culture solution was centrifuged at 40,000 g for 20 minutes, and ethanol was added to obtained supernatant so the final concentration to be 70% or more to precipitate polysaccharides, subsequently, which was centrifuged at 40,000 g for 20 minutes, and lyophilized to obtain β-glucan.

### Comparative Example 1: Aureobasidium pullulans AFO-202 Strain

Aureobasidium pullulans AFO-202 strain (FERM P-19327, Japanese Unexamined Patent Application Publication No. 2004-329077) which is generally used to produce β-glucan, was used to produce β-glucan as follows.

One of the inventors of the Japanese Unexamined Patent Application Publication No. 2004-329077 participates in the present invention, and “FO-68 strain” and “AFO-202 strain” described in the Japanese Unexamined Patent Application Publication No. 2004-329077 are the same strain.

### (1) Production of Inoculum

AFO-202 strain was inoculated on PDA plating medium and cultured at 25° C. for 5 to 7 days. Then, it was inoculated in a liquid culture medium and cultured at 25° C. for 48 hours with shaking at 125 rpm. Subsequently, a flask in which bacteria grew well was selected, and bacteria from well grown part was further inoculated into the liquid culture medium, and was cultured at 25° C. for 72 hours with shaking at 125 rpm.

### (2) Primary Culture

400 mL of the inoculum liquid was added into a culture medium containing 300 L of manufacturing water, 0.6 kg of rice bran, 3 kg of glucose, 0.9 kg of ascorbic acid, and 0.15 L of food-grade defoaming agent, adjusted pH 5.0 to 5.4 using 5% sodium hydroxide aqueous solution and 25% hydrochloric acid, which was cultured aerated and agitated for 48 hours.

### (3) Secondary Culture

200 L of the primary culture liquid was added into a culture medium containing 8,900 L of manufacturing water, 17.8 kg of rice bran, 89 kg of glucose, 26.7 kg of ascorbic acid, and 4.45 L of food-grade defoaming agent, adjusted pH 5.0 to 5.4 using 5% sodium hydroxide aqueous solution and 25% hydrochloric acid, which was cultured aerated and agitated for 72 hours.

β-glucan was purified in the same manner as above, from obtained culture solution.

### (4) Analysis of β-Glucan

Chemical structure of the β-glucan produced using AFO-202 strain was analyzed as in Example 1-(2). As a result, as shown below, it was completely different from the chemical structure of the β-glucan produced by APNN-M163 strain according to the present invention.

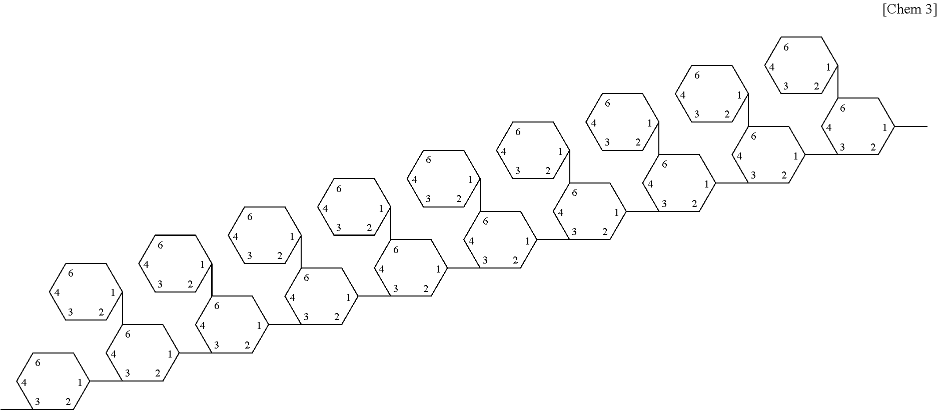

TABLE 2

| Biding position(s) | Ratio |
|---|---|
| Glc 1−> | 9 |
| −>3 Glc 1−> | 1 |
| −>3, 6 Glc 1−> | 9 |

### Test Example 1: Adhesion Test

In a flat-bottom 96-well microplate for cell culture, 2 μg/mL solution of β-glucan produced in Example 1, Example 2, or Comparative Example 1 dissolved in PBS was dispensed by 50 μL/well, which was incubated at 37° C. for 1 hour. Each well was then washed five times with 0.05% solution of nonionic surfactant (“Tween 20”) dissolved in PBS. After washing, 300 UL of PBS solution of 2% BSA-Tween 20 was added to each well, they were left to stand at room temperature for 1 hour, subsequently each well was coated by washing four times with PBS solution of Tween 20.

50 μL of biotin-labeled anti-rabbit BGIgG antibody diluted 32,000-fold or 16,000-fold was dispensed into each well and was reacted for 1 hour at room temperature. Then, 50 μL of peroxidase (POD)-labeled streptavidin diluted 5,000-fold was dispensed into each well and reacted for 30 minutes at room temperature. After washing each well 10 times with PBS solution of Tween 20, 50 μL of chromogenic liquid (TMB) was dispensed into each well and reacted for 10 to 12 minutes at room temperature.

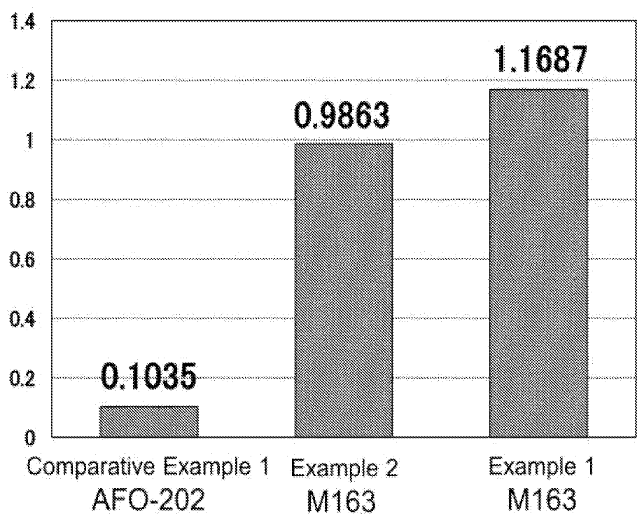

After stopping the reaction by adding 50 μL of 0.5 M HCl, absorbance (O.D.) at 450 nm was measured using a microplate reader. The results are shown in FIG. 1.

As the results shown in FIG. 1, the β-glucan produced by M163 strain according to the present invention exhibited obviously higher adhesive properties than β-glucan produced by AFO-202 strain. Presuming based on calibration curve, it is estimated that the β-glucan derived from AFO-202 strain hardly adhered to the plate, whereas the β-glucan derived from M163 strain adhered almost 500 times. The β-glucan derived from M163 strain has extremely high adhesion because it was impossible to measure its molecular weight by gel filtration chromatography due to its adhesion to the column.

### Test Example 2: Effect on MMP-9 Production

MMP-9 (Matrix Metalloproteinase-9) degrades extracellular matrix components such as collagen as a substrate, and it has been clarified that it is involved in inflammation, tissue re-forming, wound healing, and cytokine processing, as well as in infiltration and metastasis of cancer cells such as fibrosis in pancreatic carcinoma, lymph node metastasis in human breast adenocarcinoma, and local blood infiltration in malignant giant cell tumor of bone. Thus, effect of β-glucan on MMP-9 production was tested.

Specifically, dispersion of 50,000 cells/200 μL concentration in which oral squamous cell carcinoma was dispersed in 10% RPMI 1640 culture medium was dispensed into 96-well plates, and β-glucan of Example 2 or Comparative Example 1 was added so final concentration to be 10 μg/mL, further, phorbol-12-myristate 13-acetate (PMA) to induce MMP-9 was added so final concentration to be 100 ng/ml, which was incubated in a 5% CO₂ incubator at 37° C. for 24 or 72 hours. Supernatant was then collected and MMP-9 was quantified using an MMP-9 quantification kit (“Quantikine MMP-9 ELISA Kit” made by Funakoshi Co., Ltd.). In addition, for comparison purpose, additional experiments were conducted in the same way, except that (i) neither β-glucan nor PMA were added, and (ii) only PMA was added. The results are shown in Table 3.

TABLE 3

| PMA | β-glucan | 24 h culture | 72 h culture |
|---|---|---|---|
| − | — | 251.6 pg/mL | 1097.2 pg/mL |
| + | — | 3286.2 pg/mL | 20275.9 pg/mL |
| + | Comparative Example 1 | 2450.2 pg/mL | 15759.8 pg/mL |
| + | Example 2 | 1312.9 pg/mL | 11931.2 pg/mL |

As the results shown in Table 3, while MMP-9 is induced by PMA, β-glucan inhibits biosynthesis of MMP-9, and MMP-9 production inhibiting action of β-glucan produced by M163 strain according to the present invention was obviously more excellent compared to that of β-glucan produced by AFO-202 strain.

Therefore, it has been suggested that the β-glucan produced by the M163 strain according to the present invention is useful as an active ingredient for anticancer drug.

1. A method for producing β-1,3-1,6-glucan, comprising a step for culturing an Aureobasidium pullulans APNN-M163 strain (accession number: NITE BP-03377) in a culture medium.

2. The method according to claim 1, wherein the culture medium contains one or more carbon sources selected from the group consisting of glucose, polysaccharides containing glucose unit and fructose.

3. The method according to claim 1, wherein the culture medium contains nitrate ion salt as a nitrogen source.

4. A bacterium consisting of Aureobasidium pullulans APNN-M163 strain (accession number: NITE BP-03377).

5. A β-1,3-1,6-glucan having β-1,4 and β-1,2 bonds in addition to the β-1,3 and β-1,6 bonds.

6. The β-1,3-1,6-glucan according to claim 5, wherein the ratio of glucose units bound to adjacent glucose units at only 1st and 4th positions to total glucose units configuring the β-1,3-1,6-glucan ranges from 0.04 to 0.08.

7. The β-1,3-1,6-glucan according to claim 5, wherein the ratio of glucose units bound to adjacent glucose units at 1st, 2nd, 4th and 6th positions to total glucose units configuring the β-1,3-1,6-glucan is 0.04 or more and 0.08 or less.
